(19) United States
(12) Patent Application Publication
(10) Pub. No.: US 2017/0365769 A1
(43) Pub. Date: Dec. 21, 2017

(54) ULTRASONIC TRANSDUCER AND ULTRASONIC MEDICAL DEVICE

(71) Applicant: OLYMPUS CORPORATION, Tokyo (JP)

(72) Inventor: Hiroshi ITO, Tokyo (JP)

(73) Assignee: OLYMPUS CORPORATION, Tokyo (JP)

(21) Appl. No.: 15/690,794

(22) Filed: Aug. 30, 2017

Related U.S. Application Data
(63) Continuation of application No. PCT/JP2015/057448, filed on Mar. 13, 2015.

(51) Int. Cl. H01L 41/083; B06B 1/06; A61N 7/00

(52) U.S. Cl. CPC H01L 41/0838; A61N 7/00; B06B 1/0611

(57) ABSTRACT

An ultrasonic transducer includes: two metal blocks; a plurality of piezoelectric elements having rectangular surfaces and stacked between the metal blocks; and bonding materials bonding the metal block and the piezoelectric element and the piezoelectric elements to each other. Thermal expansion coefficients in the diagonal directions from the center of the surface of the piezoelectric element to the four corners thereof are equal to each other.

### CROSS-REFERENCE TO RELATED APPLICATIONS

This application is based on PCT/JP2015/057448 filed on Mar. 13, 2015. The content of the PCT application is incorporated herein by reference.

### BACKGROUND OF THE INVENTION AND RELATED ART STATEMENT

The present invention relates to an ultrasonic transducer that excites an ultrasonic wave and an ultrasonic medical device.

An ultrasonic treatment instrument that performs coagulation/incision treatment of biological tissues using ultrasonic vibration incorporates a bolt-clamped Langevin transducer in a handpiece as an ultrasonic vibration source. In the bolt-clamped Langevin transducer, a piezoelectric element that converts an electric signal into mechanical vibration is held between front and back masses which are metal members and firmly clamped by a bolt to be integrated with the masses, whereby the entire transducer structure is integrally transduced. A transducer in which a piezoelectric element is held between metal members, integrated therewith by some means, including an adhesive, and transduced integrally therewith is called “Langevin transducer”, and a Langevin transducer in which the piezoelectric element is integrated with the metal members by a bolt is called “bolt-clamped Langevin transducer”. Typically, the bolt-clamped Langevin transducer uses lead zirconate titanate (PZT, Pb(Zrₓ, Ti₁₋ₓ)O₃) as the piezoelectric element, the piezoelectric element is formed into a ring shape, and a bolt is pushed into the hole of the ring.

The PZT has excellent characteristics, such as high productivity and high electromechanical conversion efficiency, as a piezoelectric material and has found applications in various fields of ultrasonic transducers and actuators over many years. In recent years, however, lead zirconate titanate (PZT), which contains lead that has a bad influence on the environment, is demanded to be replaced by a lead-free piezoelectric material.

As a lead-less piezoelectric material having high electromechanical conversion efficiency, lithium niobate (LiNbO₃) of a piezoelectric single crystal is known. As a method for producing a Langevin transducer using lithium niobate at low cost, there is known a method that bonds a metal block and a piezoelectric element for integration, and particularly, when they are bonded by means of a brazing material such as a solder, more satisfactory vibration characteristics can be obtained than when bonded by means of an adhesive. However, the bonding using the brazing material typically requires a high-temperature process. The high-temperature process may cause crack of the piezoelectric element by thermal stress at a dissimilar material bonding part where the metal block and the piezoelectric element are bonded together.

As a method for alleviating stress generated at the dissimilar material bonding part between the metal block and the piezoelectric element to prevent crack of the piezoelectric element, a method that forms a groove or a recess in the metal block is disclosed in JP 2008-128875A.

### SUMMARY OF INVENTION

An ultrasonic transducer according to an aspect includes: two metal blocks; a plurality of piezoelectric elements having rectangular surfaces and stacked between the metal blocks; and bonding materials bonding the metal block and the piezoelectric element and the piezoelectric elements to each other. Thermal expansion coefficients in the diagonal directions from the center of the surface of the piezoelectric element to the four corners thereof are equal to each other.

An ultrasonic medical device according to another aspect includes: the ultrasonic transducer; and a probe distal end part receiving ultrasonic vibration generated in the ultrasonic transducer and treating a body tissue.

### BRIEF DESCRIPTION OF DRAWINGS

FIGS. 1A and 1B each illustrate an ultrasonic transducer according to an embodiment;

FIG. 2 illustrates the crystal axes of a piezoelectric single crystal material according to the present embodiment and the coordinate system of a wafer W;

FIG. 3 is the coordinate system of the wafer W of the ultrasonic transducer according to the present embodiment;

FIGS. 4A and 4B each illustrate the ultrasonic transducer according to the another embodiment;

FIG. 5 illustrates a piezoelectric element according to a first embodiment;

FIGS. 6A and 6B illustrate the relationship between the crystal axes of lithium niobate and the coordinate system of a wafer W of the piezoelectric element according to the first embodiment;

FIG. 7 illustrates a thermal expansion coefficient corresponding to the Euler angle of the lithium niobate;

FIG. 8 illustrates how to cut out the piezoelectric element according to the first embodiment from 36-degree rotation Y-cut X-propagation lithium niobate;

FIG. 9 illustrates a piezoelectric element according to a second embodiment;

FIG. 10 illustrates a thermal expansion coefficient corresponding to the Euler angle of the lithium niobate;

FIG. 11 illustrates how to cut out the piezoelectric element according to the second embodiment from the 36-degree rotation Y-cut X-propagation lithium niobate;

FIG. 12 illustrates a thermal expansion coefficient corresponding to the Euler angle of lithium tantalite;

FIG. 13 illustrates the entire configuration of an ultrasonic medical device according to the present embodiment;

FIG. 14 illustrates the schematic entire configuration of a transducer unit of the ultrasonic medical device according to the present embodiment; and

FIG. 15 illustrates the entire configuration of an ultrasonic medical device according to another aspect of the ultrasonic medical device according to the present embodiment.

### DESCRIPTION OF EMBODIMENTS

Hereinafter, an ultrasonic transducer 1 according to an embodiment will be described.

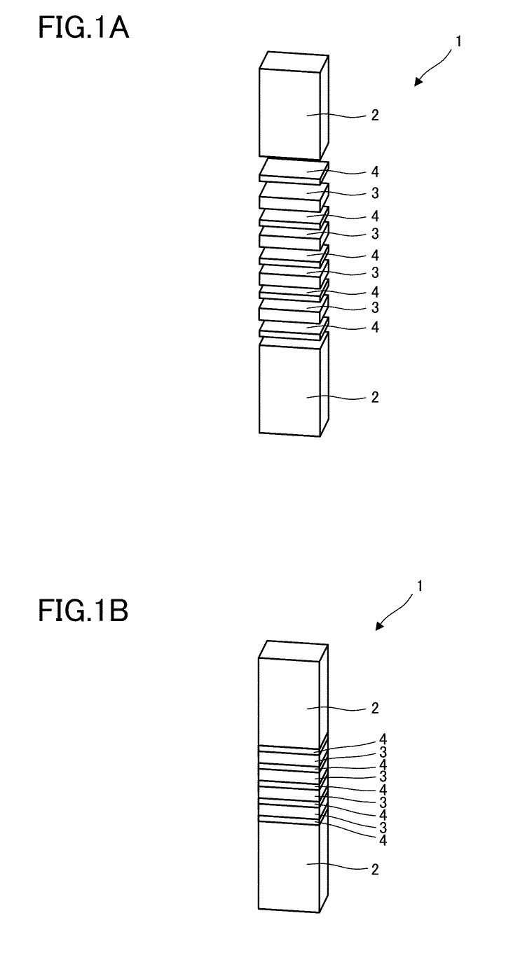

FIGS. 1A and 1B each illustrate the ultrasonic transducer 1 according to the present embodiment. FIG. 1A illustrates the ultrasonic transducer 1 according to the present embodiment before bonding. FIG. 1B illustrates the ultrasonic transducer 1 according to the present embodiment after bonding.

As illustrated in FIG. 1A, the ultrasonic transducer 1 according to the present embodiment includes two metal blocks 2, a plurality of piezoelectric elements 3 stacked between the metal blocks 2, and bonding materials 4 each bonding the metal block 2 and the piezoelectric element 3 and the piezoelectric elements 3 to each other.

The metal block 2, insulating member 5, and piezoelectric element 3, and the piezoelectric elements 3 are tightly bonded together by the bonding material 4 as illustrated in FIG. 1B. The bonding process may be achieved by heating up to the melting temperature of the bonding material 4, followed by cooling.

The materials of the ultrasonic transducer 1 according to the present embodiment will be described individually.

As the piezoelectric elements 3, single crystal lithium niobate (LiNbO₃) having a high Curie point is used. For example, preferably a lithium niobate wafer having a crystal orientation called 36-degree rotation Y-cut is used so as to make large an electromechanical coupling coefficient in the thickness direction of the piezoelectric element 3. A base metal such as Ti/Pt or Cr/Ni/Au is formed on both the front and back surfaces of the lithium niobate wafer so as to improve wettability and adhesion between the lithium niobate and a lead-free solder, followed by, e.g., dicing into rectangular pieces. The adjacent piezoelectric elements 3 are stacked with their upper and lower surfaces reversed to each other.

As the bonding material 4, a lead-free solder having a melting point lower than the Curie point, preferably, a melting point equal to or lower than half of the Curie point is used. However, when the solder is used as the bonding material and supplied in the form of solder pellets, it is difficult to bond a part having an uneven shape without bubbles. Thus, the bonding parts between the piezoelectric element 3 and the metal block 2, and between the piezoelectric elements 3 preferably each have a flat surface. The thickness of the bonding material 4 may be determined considering the distance between the above members after bonding.

The metal block 2 is formed of materials having different thermal expansion coefficients selected from among an aluminum alloy such as duralumin, a titanium alloy such as 64Ti, pure titanium, stainless steel, soft steel, nickel-chrome steel, tool steel, brass, and monel metal.

The ultrasonic transducer 1 formed as illustrated in FIG. 1B is attached, at its side, with a flexible printed circuit connected to an unillustrated electric cable. Further, like general ultrasonic transducers, positive and negative electrode layers are alternately attached to both ends and between the stacked piezoelectric elements 3. Application of a driving electric signal to the piezoelectric elements 3 allows the ultrasonic transducer 1 to be driven.

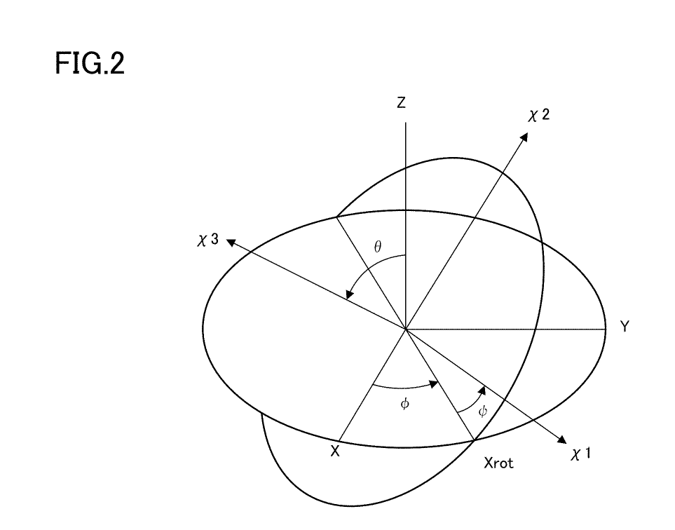

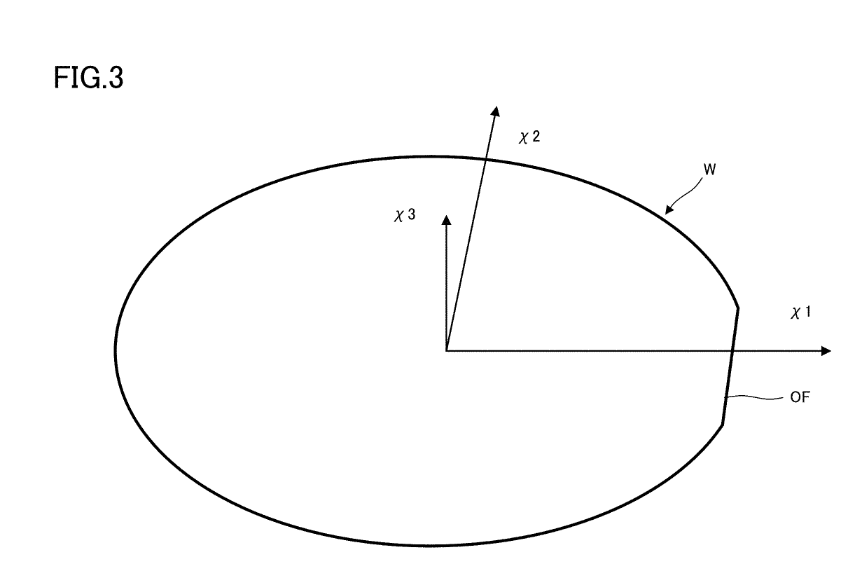

FIG. 2 illustrates the crystal axes of the piezoelectric single crystal material according to the present embodiment and the coordinate system of a wafer W. FIG. 3 is the coordinate system of the wafer W of the ultrasonic transducer 1 according to the present embodiment.

The piezoelectric single crystal material is an anisotropic material and thus has different thermal expansion coefficients in different directions. However, when the material is rotated with the direction perpendicular to the surface of the piezoelectric element 3 as its rotation axis, the thermal expansion coefficient of the piezoelectric element 3 in the in-plane direction periodically fluctuates, with the result that the same thermal expansion coefficient maybe obtained in four directions. When the aspect ratio of the outer shape and the orientation thereof with respect to the crystal axes are selected so as to make the four corners of the rectangular piezoelectric element 3 coincide with the four directions, it is possible to make thermal expansion coefficients equal to each other in the diagonal directions of the rectangular piezoelectric element 3.

The crystal axes (X, Y, Z) of the piezoelectric single crystal material of FIG. 2 and the coordinate system (χ¹, χ², χ³) set on the wafer W of FIG. 3 cut from the piezoelectric single crystal material are associated with each other by three consecutive rotations, and the rotation angles thereof are called Euler angles.

As illustrated in FIG. 3, in the coordinate system on the wafer W, the direction vertical to the surface of the wafer W is assumed to be +χ³, the direction orthogonal to an orientation flat OF indicating the directions of the crystal axes from the center of the wafer W is assumed to be +χ¹, and the direction of +χ² is set so that (χ¹, χ², χ³) forms a right-hand system.

First, the crystal axes (X, Y, Z) are considered. The first rotation is a rotation about the Z-axis by an angle φ. Here, a positive rotation direction is defined as the rotation direction in which a right-hand screw advances in the rotation axis positive direction. The same is applied to the following two rotations. The angle φ can be set in a range of 0° to 360°. By the first rotation, the original X-axis is converted into χ′. The second rotation is a rotation about the axis newly defined as χ′, and the rotation angle is θ. This rotation is limited within a range of 0° to 180°. By the second rotation, the Z-axis is converted into the coordinate axis called χ³ which is vertical to the surface of the wafer W. The third rotation is a rotation about the χ³ axis, and the rotation angle is ψ. The angle ψ can be set in a range of 0° to 360°, and the χ^{rot} axis is converted into the χ¹ axis which extends vertically to the orientation flat OF of the wafer W. The wafer W surface is thus determined by the rotation angles φ and θ, and a direction in the wafer W surface is determined by the rotation angle ψ.

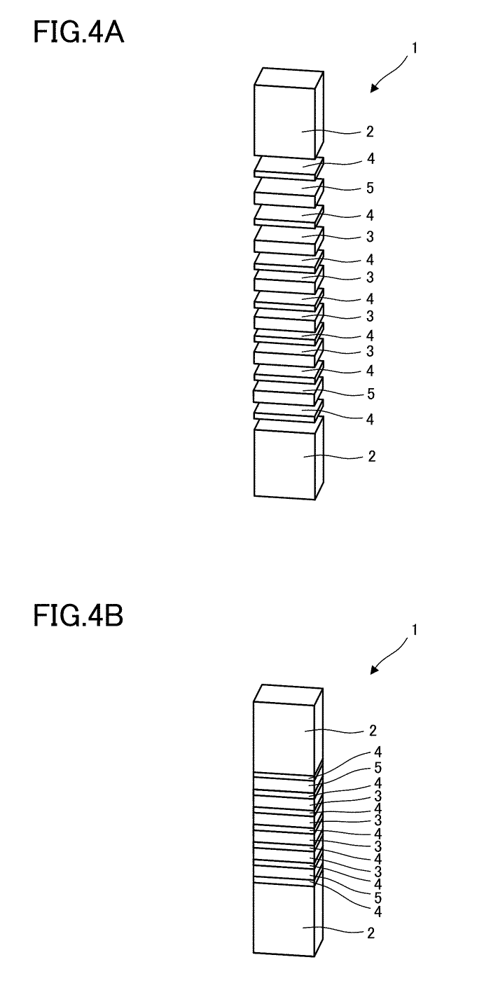

FIGS. 4A and 4B each illustrate an ultrasonic transducer 1 according to another embodiment. FIG. 4A illustrates the ultrasonic transducer 1 according to the another embodiment before bonding. FIG. 4B illustrates the ultrasonic transducer 1 according to the another embodiment after bonding.

As illustrated in FIG. 4A, the ultrasonic transducer 1 according to the another embodiment includes two metal blocks 2, a plurality of piezoelectric elements 3 stacked between the metal blocks 2, bonding materials 4 each bonding the metal block 2 and the piezoelectric element 3 together and piezoelectric elements 3 together, and an insulating member 5 having high insulating performance. That is, the insulating member 5 is newly provided between the metal block 2 and the piezoelectric element 3.

The metal block 2, insulating member 5, and piezoelectric element 3, and the piezoelectric elements 3 are tightly bonded together by the bonding material 4 as illustrated in FIG. 4B. The bonding process may be achieved by heating to the melting temperature of the bonding material 4, followed by cooling.

The piezoelectric element 3 and bonding material 4 of the ultrasonic transducer 1 according to the another embodiment are made of the same materials as those of the respective piezoelectric element 3 and bonding material 4 of the ultrasonic transducer 1 illustrated in FIGS. 1A and 1B. The insulating member 5 is preferably made of alumina or zirconia having an insulating property and high mechanical strength.

The ultrasonic transducer 1 formed as illustrated in FIG. 4B is attached, at its side, with a flexible printed circuit connected to an unillustrated electric cable. Further, like general ultrasonic transducers, positive and negative electrode layers are alternately attached to both ends and between the stacked piezoelectric elements 3. Application of a driving electric signal to the piezoelectric elements 3 allows the ultrasonic transducer 1 to be driven.

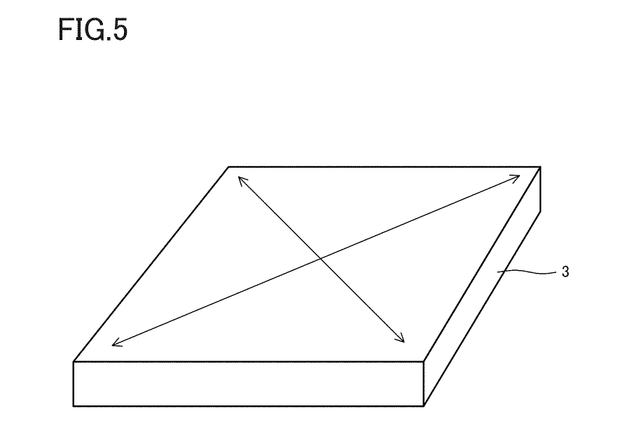

FIG. 5 illustrates a piezoelectric element 3 according to a first embodiment.

The piezoelectric element 3 according to the first embodiment has, for example, a square shape and formed so as to make the thermal expansion coefficients equal to each other in the diagonal directions on the surface thereof. For example, as the piezoelectric element 3 of the first embodiment, a lithium niobate wafer having a crystal orientation called 36-degree rotation Y-cut X-propagation is used. The 36-degree rotation Y-cut X-propagation is expressed as (180°, 54°, 180°) in terms of Euler angle coordinates assuming that φ, θ, and ψ in FIG. 2 are set to 180°, 54°, and 180°, respectively.

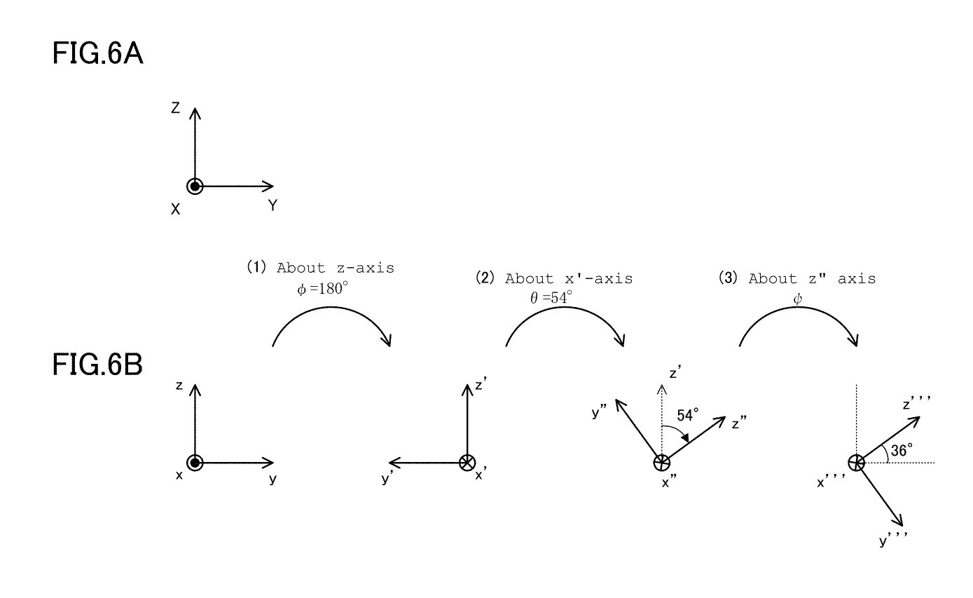

FIGS. 6A and 6B illustrate the relationship between the crystal axes of the lithium niobate and the coordinate system of a wafer W of the piezoelectric element 3 according to the first embodiment. FIG. 6A illustrates the crystal axes of the lithium niobate, and FIG. 6B illustrates a state where the crystal axes of the lithium niobate are converted into the coordinate system of the wafer W.

First, rotation is made by an angle of φ=180° about the Z-axis on the coordinate system of FIG. 6B corresponding to the coordinate axes of the lithium niobate illustrated in FIG. 6A. Subsequently, rotation is made by an angle of θ=54° about the x′ axis to determine the wafer surface. Then, rotation is made by an angle of ψ=180° about the z″ axis to determine a wafer in-plane direction.

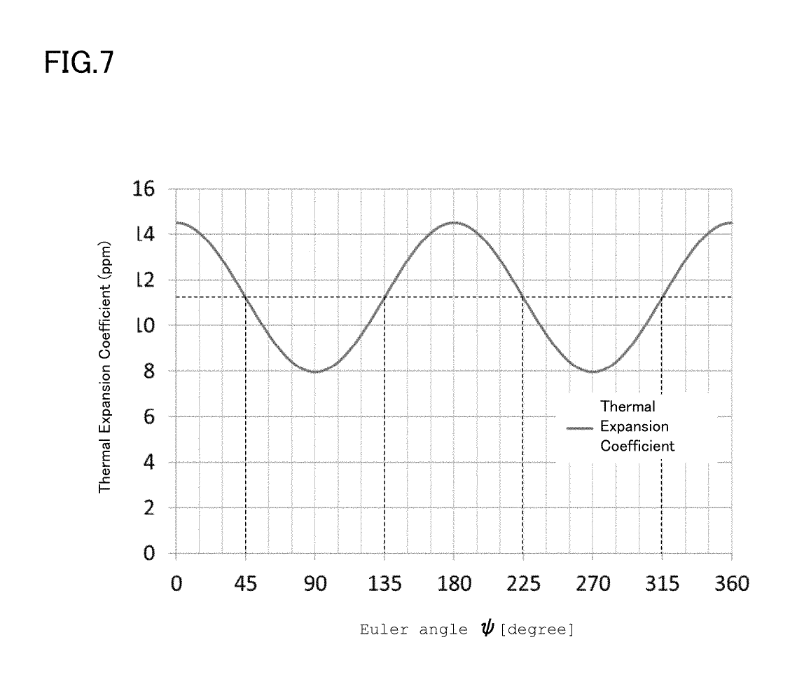

FIG. 7 illustrates a thermal expansion coefficient corresponding to the Euler angle of the lithium niobate.

The horizontal axis of FIG. 7 indicates the angle ψ of the third rotation of a 36-degree Y-cut substrate in terms of Euler angle coordinates. It can be seen from the graph that there are four Euler angles having the same thermal expansion coefficient in a range of thermal expansion coefficient of 8 ppm to 14.5 ppm. Particularly, at the Euler angles ψ of 45°, 135°, 225°, and 315°, the same thermal expansion coefficient can be obtained every 90 degrees, so that when the thermal expansion coefficients are made equal in the diagonal directions of the piezoelectric element, the piezoelectric element is formed into a square shape, which is the most favorable shape.

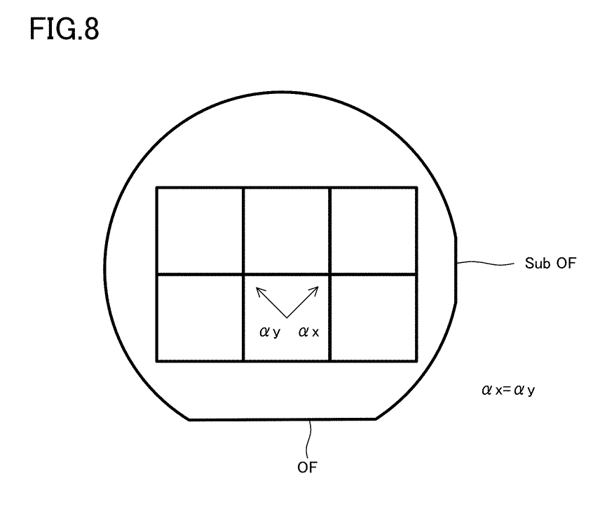

FIG. 8 illustrates how to cut out the piezoelectric element 3 according to the first embodiment from the 36-degree rotation Y-cut X-propagation lithium niobate.

To obtain the piezoelectric element 3 having a shape as illustrated in FIG. 5 from a lithium niobate 36-degree Y-cut X-propagation substrate, the piezoelectric element 3 may be cut by dicing in both the directions parallel and vertical to the orientation flat OF, as illustrated in FIG. 8. At this time, the sides of the piezoelectric element 3 are parallel to the parallel and vertical directions of the X-axis of the crystal axes. When the piezoelectric element 3 is thus cut so that directions corresponding to the Euler angles ψ=45°, 135°, 225°, and 315° in the lithium niobate 36-degree Y-cut X-propagation substrate form the diagonal lines, it is possible to obtain the square piezoelectric element 3 in which the thermal expansion coefficients in the diagonal directions αx and αy are equal to each other. Thus, when the obtained piezoelectric element 3 is bonded to the insulating member 5 or metal block 2 which is an isotropic material, thermal stresses generated at the four corners of the piezoelectric element 3 can be made equal. Since the thermal stresses generated at the four corners are equal, it is possible to uniformly reduce the thermal stresses generated at the four corners where stress is likely to concentrate by adequately setting the thermal expansion coefficient of the insulating member 5 or metal bock 2, thereby making it possible to reduce crack of the piezoelectric element 3.

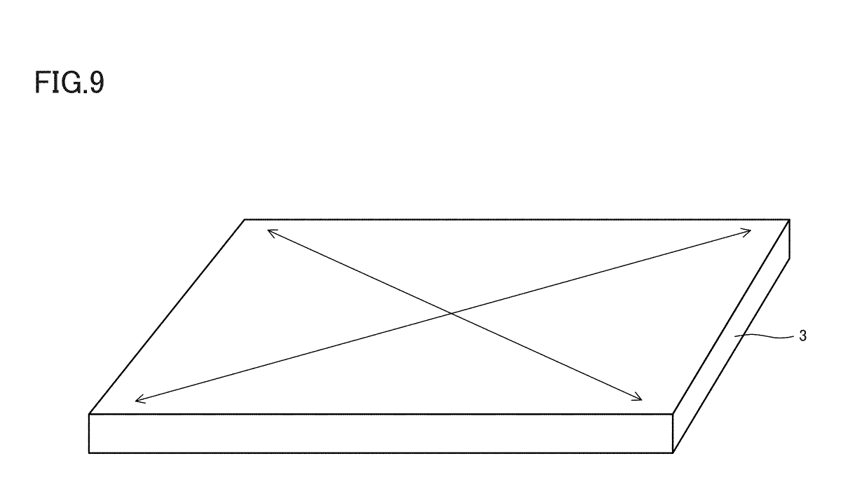

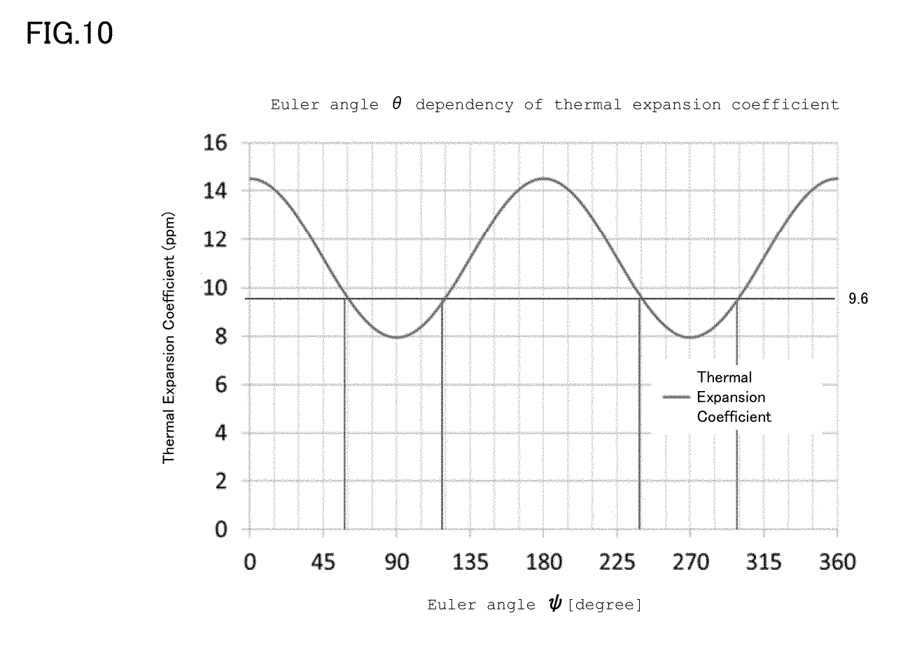

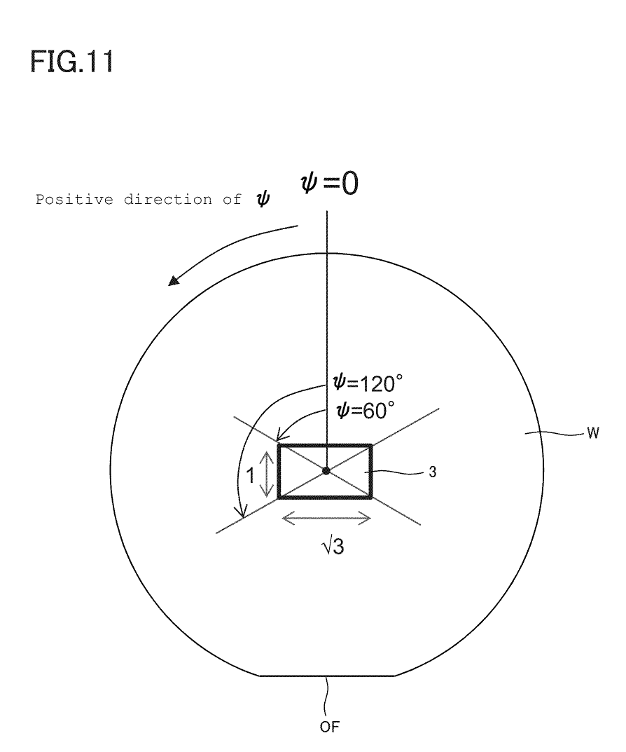

FIG. 9 illustrates a piezoelectric element 3 according to a second embodiment. FIG. 10 illustrates a thermal expansion coefficient corresponding to the Euler angle of the lithium niobate. FIG. 11 illustrates how to cut out the piezoelectric element 3 according to the second embodiment from the 36-degree rotation Y-cut X-propagation lithium niobate.

The piezoelectric element 3 according to the second embodiment has a rectangular shape and formed so as to make the thermal expansion coefficients equal to each other in the diagonal directions on the surface thereof. For example, as the piezoelectric element 3 of the second embodiment, a lithium niobate wafer having a crystal orientation called 36-degree rotation Y-cut X-propagation is used. As illustrated in FIG. 10, in the 36-degree rotation Y-cut X-propagation lithium niobate wafer, the same thermal expansion coefficient (9.6 ppm) is obtained at the Euler angles of ψ=60°, 120°, 240°, and 300° in the third rotation illustrated in FIG. 2.

Thus, as illustrated in FIG. 11, assuming that a direction vertical to the orientation flat OF from the center of the piezoelectric element 3 is 0°, the piezoelectric element 3 is preferably cut such that the directions of the four corners from the center of the piezoelectric element 3 are 60°, 120°, 240°, and 300° in the counterclockwise direction.

The piezoelectric element 3 cut out is a rectangle whose short side extends in a direction vertical to the orientation flat OF and whose long side extends in a direction parallel to the orientation flat OF. The ratio between the short and long sides is 1:√3.

When the piezoelectric element 3 is thus cut from the lithium niobate 36-degree Y-cut X-propagation substrate, it is possible to obtain the rectangular piezoelectric element 3 in which the thermal expansion coefficients in the diagonal directions are equal to each other. Thus, when the obtained piezoelectric element 3 is bonded to the insulating member 5 or metal block 2 which is an isotropic material, thermal stresses generated at the four corners of the piezoelectric element 3 can be made equal. Since the thermal stresses generated at the four corners are equal, it is possible to uniformly reduce the thermal stresses generated at the four corners by adequately setting the thermal expansion coefficient of the insulating member 5 or metal block 2, thereby making it possible to reduce a possibility of occurrence of crack in the piezoelectric element 3.

In the piezoelectric elements 3 according to the first and second embodiments, the thermal expansion coefficients are made equal to each other in the diagonal directions; however, the diagonal directions need not be completely equal to the Euler angles, and a slight error is allowed. For example, an error of the Euler angle ψ is preferably within ±4°, because a difference between the thermal expansion coefficients in the diagonal directions can be reduced to 1 ppm or less. Therefore, in the embodiments, the diagonal direction may include a direction within ±4° with respect to the diagonal line.

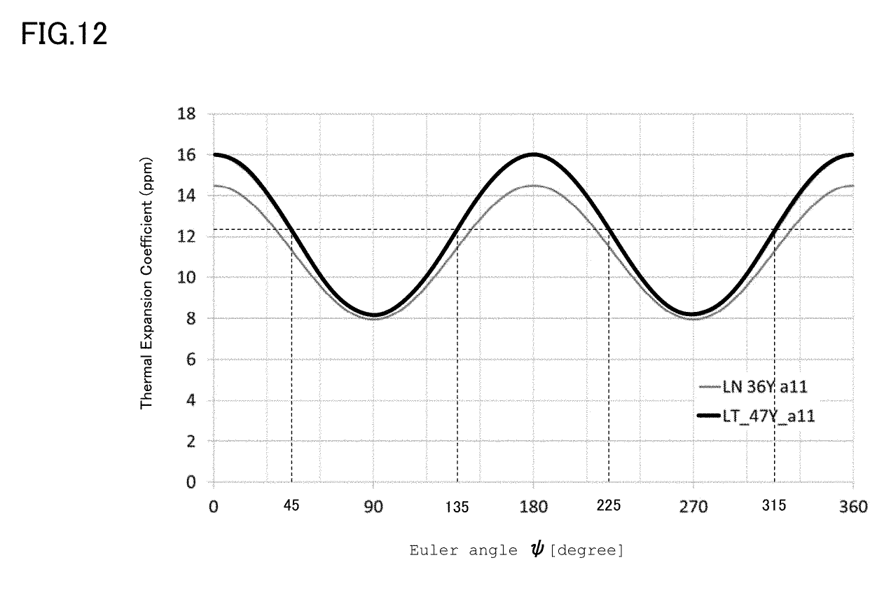

FIG. 12 illustrates a thermal expansion coefficient corresponding to the Euler angle of lithium tantalate.

Although the lithium niobate is used as a material for the piezoelectric element 3, a different material may be used. For example, the Euler angle dependence of the thermal expansion coefficient of 47-degree rotation Y-cut X-propagation (180°, 53°, ψ) lithium tantalate (LiTaO3) is shown with the thick curve in FIG. 12. The thin curve is the thermal expansion coefficient of 36-degree rotation Y-cut X-propagation (180°, 54°, ψ) lithium niobate corresponding to the Euler angle.

In the lithium tantalate 47-degree rotation Y-cut X-propagation, the same thermal expansion coefficient (12.1 ppm) is obtained at the Euler angles of ψ=45°, 135°, 225°, and 315° in the third rotation. That is, when the piezoelectric element 3 is thus cut from the wafer W by dicing so that directions corresponding to the Euler angles ψ=45°, 135°, 225°, and 315° form the diagonal lines, it is possible to obtain the square piezoelectric element 3 in which the thermal expansion coefficients in the diagonal directions are equal to each other. By changing the thermal expansion coefficients which are equal to each other, the piezoelectric element 3 can be formed into a rectangular shape.

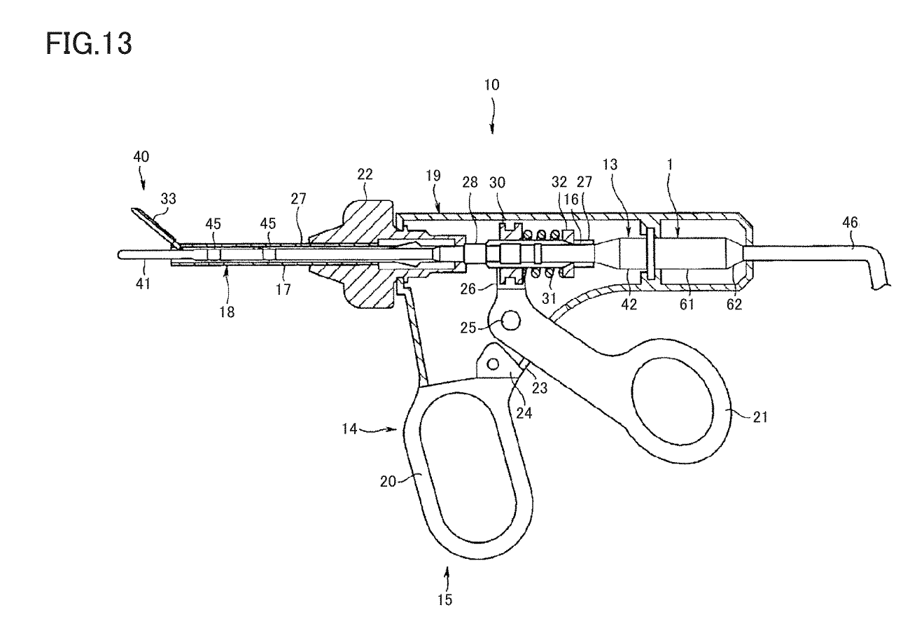

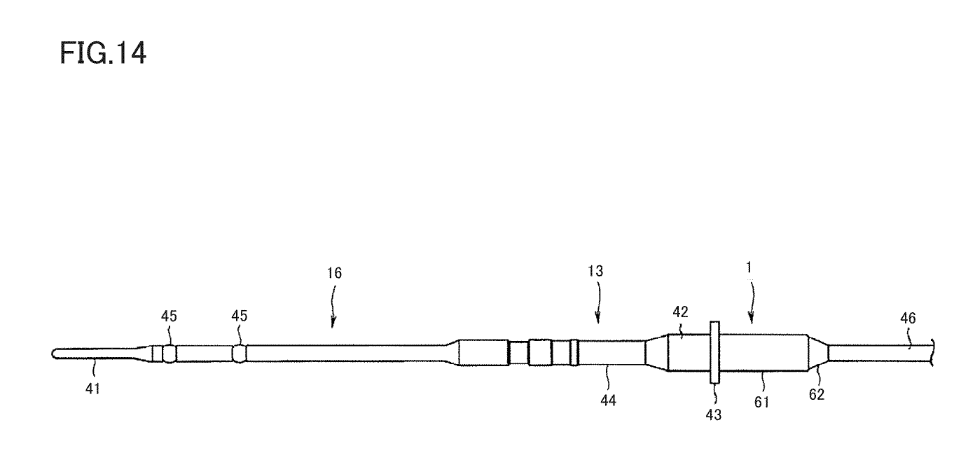

FIG. 13 illustrates the entire configuration of an ultrasonic medical device according to the present embodiment. FIG. 14 illustrates the schematic entire configuration of a transducer unit of the ultrasonic medical device according to the present embodiment.

An ultrasonic medical device 10 illustrated in FIG. 13 includes a transducer unit 13 having the ultrasonic transducer 1 that mainly generates ultrasonic vibration and a handle unit 14 for an operator to treat an affected part using the ultrasonic vibration.

The handle unit 14 includes an operation part 15, an insertion sheath part 18 constituted of a long outer tube 17, and a distal end treatment part 40. The base end portion of the insertion sheath part 18 is attached to the operation part 15 so as to be rotatable about the axis of the sheath part 18. The distal end treatment part 40 is provided at the distal end of the insertion sheath part 18. The operation part 15 of the handle unit 14 includes an operation part main body 19, a fixed handle 20, a movable handle 21, and a rotary knob 22. The operation part main body 19 is formed integrally with the fixed handle 20.

A slit 23 through which the movable handle 21 is inserted is formed on the back side of a connection portion between the operation part main body 19 and the fixed handle 20. The upper portion of the movable handle 21 is inserted through the slit 23 and extends inside the operation part main body 19. A handle stopper 24 is fixed to the lower end portion of the slit 23. The movable handle 21 is turnably attached to the operation part main body 19 through a handle spindle 25. Accompanying a turning movement of the movable handle 21 with the handle spindle 25 as the center, the movable handle 21 is opened/closed with respect to the fixed handle 20.

A substantially U-shaped connection arm 26 is provided at the upper end portion of the movable handle 21. The insertion sheath part 18 has an outer tube 17 and an operation pipe 27 inserted into the outer tube 17 so as to be movable in the axial direction of the outer tube 17. A large diameter portion 28 larger in diameter than a distal end side portion is formed at the base end portion of the outer tube 17. The rotary knob 22 is fitted around the large diameter portion 28.

A ring-shaped slider 30 is provided on the outer peripheral surface of the operation pipe 27 so as to be movable in the axial direction of the operation pipe 27. On the back side of the slider 30, a fixed ring 32 is provided through a coil spring (elastic member) 31.

Further, a base end portion of a holding part 33 is turnably connected to the distal end portion of the operation pipe 27 through a working pin. The holding part 33 constitutes, together with a distal end part 41 of a probe 16, the treatment part of the ultrasonic medical device 10. When the operation pipe 27 is moved in the axial direction, the holding part 33 is pushed/pulled in the front-back direction through the working pin. At this time, when the operation pipe 27 is moved to an operator's hand side, the holding part 33 is turned about a fulcrum pin in the counterclockwise direction through the working pin. As a result, the holding part 33 is turned in a direction approaching the distal end part 41 of the probe 16 (closing direction). At this time, a body tissue can be held between the cantilever holding part 33 and the distal end part 41 of the probe 16.

In a state where the body tissue is thus held, an electric power is supplied from an ultrasonic power supply to the ultrasonic transducer 1 to transduce the ultrasonic transducer 1. This ultrasonic vibration is transmitted to the distal end part 41 of the probe 16. Then, the ultrasonic vibration is used to treat the body tissue held between the holding part 33 and the distal end part 41 of the probe 16.

As illustrated in FIG. 14, the transducer unit 13 is a unit obtained by integrally assembling the ultrasonic transducer 1 and the probe 16 which is a rod-like vibration transmission member that transmits the ultrasonic vibration generated in the ultrasonic transducer 1.

A horn 42 that amplifies the amplitude of the ultrasonic vibration is connected to the ultrasonic transducer 1. The horn 42 is formed of duralumin, stainless steel, or a titanium alloy such as 64Ti (Ti-6Al-4V). The horn 42 is formed into a cone shape having an outer diameter reduced toward the distal end thereof and has an outward flange 43 on the base end outer peripheral portion thereof. The shape of the horn 42 is not limited to the cone shape, but may be an exponential shape having an outer diameter exponentially reduced toward the distal end thereof or a step shape having an outer diameter reduced stepwise toward the distal end thereof.

The probe 16 has a probe main body 44 formed of a titanium alloy such as 64Ti (Ti-6Al-4V). On the distal end side of the probe main body 44, the ultrasonic transducer 1 connected to the horn 42 is provided. In such a manner as described above, the transducer unit 13 integrally including the probe 16 and ultrasonic transducer 1 is formed. In the probe 16, the probe main body 44 and the horn 42 are threadably connected to each other, and the probe main body 44 and the horn 42 are bonded to each other.

The ultrasonic vibration generated in the ultrasonic transducer 1 is amplified by the horn 42 and is then transmitted to the distal end part 41 of the probe 16. A treatment part to be described later for treating the body tissue is formed at the distal end part 41 of the probe 16.

Further, on the outer peripheral surface of the probe main body 44, two ring-shaped rubber linings 45 formed of an elastic member are fitted to several locations of a vibration node position, which is on the midway in the axial direction of the probe main body 44, so as to be spaced apart from each other. These rubber linings 45 prevent contact between the outer peripheral surface of the probe main body 44 and the operation pipe 27 to be described later. That is, in the course of the assembly of the insertion sheath part 18, the probe 16 as a transducer-integrated probe is inserted inside the operation pipe 27. At this time, the rubber linings 45 prevent contact between the outer peripheral surface of the probe main body 44 and the operation pipe 27.

Further, the ultrasonic transducer 1 is electrically connected, through an electric cable 46, to an unillustrated power supply device body that supplies current for use in generating the ultrasonic vibration. Supplying electric power from the power supply device body to the ultrasonic transducer 1 through wiring in the electric cable 46 allows the ultrasonic transducer 1 to be driven. The transducer unit 13 includes the ultrasonic transducer 1 that generates the ultrasonic vibration, the horn 42 that amplifies the generated ultrasonic vibration, and the probe 16 that transmits the amplified ultrasonic vibration.

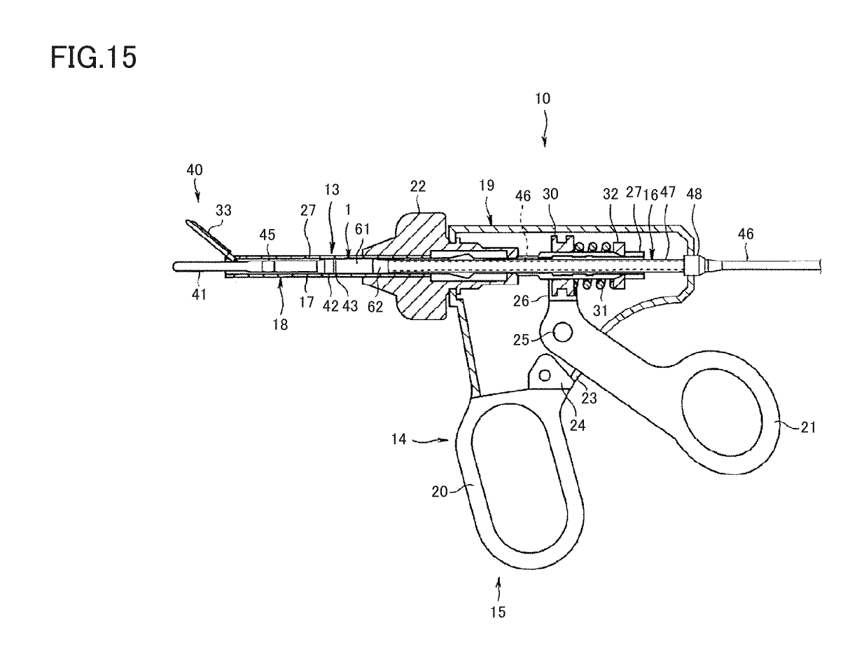

FIG. 15 illustrates the entire configuration of an ultrasonic medical device according to another aspect of the ultrasonic medical device according to the present embodiment.

The ultrasonic transducer 1 and the transducer unit 13 may not necessarily be housed inside the operation part main body 19 as illustrated in FIG. 13, but may be housed inside the operation pipe 27 as illustrated in FIG. 15. In the ultrasonic medical device 10 of FIG. 15, the electric cable 46 extending between a bending stopper 62 of the ultrasonic transducer 1 and a connector 48 provided at the base portion of the operation part main body 19 is inserted through a metal pipe 47 and housed therein. The connector 48 is not essential, but, instead, a configuration maybe adopted in which the electric cable 46 is extended up to the inside of the operation part main body 19 and is connected to the bending stopper 62 of the ultrasonic transducer 1. The configuration of the ultrasonic medical device 10 as illustrated in FIG. 15 can further save the interior space of the operation part main body 19. The function of the ultrasonic medical device 10 of FIG. 15 is the same as that of the ultrasonic medical device 10 of FIG. 13, so detailed descriptions thereof will be omitted.

As described above, the ultrasonic transducer 1 according to the present embodiment includes the two metal blocks 2, the plurality of piezoelectric elements 3 having rectangular surfaces and stacked between the metal blocks 2, the bonding materials 4 each bonding the metal block 2 and the piezoelectric element 3 and the piezoelectric elements 3 to each other. In the thus configured ultrasonic transducer 1, the thermal expansion coefficients in the diagonal directions from the center of the surface of the piezoelectric element 3 to the four corners thereof are equal to each other, so that thermal stresses generated at the four corners of the rectangular piezoelectric element can be made close to equal, thereby making it possible to reduce crack.

Further, according to the ultrasonic transducer 1 of the present embodiment, the piezoelectric element 3 is cut, from the 36-degree rotation Y-cut X-propagation lithium niobate wafer, into a shape having sides parallel and vertical to the X-axis of the crystal axes and can thus be cut out properly.

Further, according to the ultrasonic transducer 1 of the present embodiment, the surface of the piezoelectric element 3 has a square shape, so that thermal stresses generated at the four corners of the piezoelectric element can be made equal to each other.

Further, according to the ultrasonic transducer 1 of the present embodiment, the insulating member 5 stacked between the metal block 2 and the piezoelectric element 3 is provided, making it possible to properly operate the transducer.

Further, the ultrasonic medical device 10 according to the present embodiment includes the ultrasonic transducer 1 and the probe distal end part receiving the ultrasonic vibration generated in the ultrasonic transducer 1 and treating the body tissue. Thus, there can be provided an ultrasonic medical device 10 with a reduced stress and excellent vibration transmission efficiency.

The present invention is not limited to the above embodiments. That is, in describing the embodiments, many specific details are included for illustrative purpose; however, a person skilled in the art can understand that the details added with variations or modifications do not exceed the scope of the present invention. Therefore, the illustrative embodiments of the present invention have been described without causing the claimed invention to lose generality and without imposing any limitation thereon.

### REFERENCE SIGNS LIST

1: Ultrasonic transducer
2: Metal Block
3: Piezoelectric element
4: Bonding material
5: Insulating member

1. An ultrasonic transducer comprising:
two metal blocks;
a plurality of piezoelectric elements having rectangular surfaces and stacked between the metal blocks; and
bonding materials bonding the metal block and the piezoelectric element and the piezoelectric elements to each other,
wherein
thermal expansion coefficients in the diagonal directions from the center of the surface of the piezoelectric element to the four corners thereof are equal to each other.

2. The ultrasonic transducer according to claim 1,
wherein
the piezoelectric elements made of a 36-degree rotation Y-cut X-propagation lithium niobate wafer, have sides parallel and vertical to the X-axis of the crystal axes of the lithium niobate wafer.

3. The ultrasonic transducer according to claim 1,
wherein
the surface of the piezoelectric element has a square shape.

4. The ultrasonic transducer according to claim 1,
wherein
an insulating member stacked between the metal block and the piezoelectric element is provided.

5. An ultrasonic medical device, comprising:
the ultrasonic transducer according to claim 1; and
a probe distal end part receiving ultrasonic vibration generated in the ultrasonic transducer and treating a body tissue.
